(19) United States
(12) Patent Application Publication
(10) Pub. No.: US 2023/0399293 A1
(43) Pub. Date: Dec. 14, 2023

(54) METHOD FOR PRODUCING FLUORINE-CONTAINING COMPOUND AND FLUORINE-CONTAINING COMPOUND

(71) Applicant: AGC Inc., Tokyo (JP)

(72) Inventors: Toyokazu ENTA, Chiyoda-ku (JP); Makoto UNO, Chiyoda-ku (JP)

(73) Assignee: AGC Inc., Tokyo (JP)

(21) Appl. No.: 18/456,611

(22) Filed: Aug. 28, 2023

Related U.S. Application Data
(63) Continuation of application No. PCT/JP2022/008822, filed on Mar. 2, 2022.

(30) Foreign Application Priority Data
Mar. 5, 2021 (JP) 2021-034906

(51) Int. Cl. C07C 309/65; C07C 303/30; C07C 19/08; C07C 43/12

(52) U.S. Cl. CPC C07C 309/65; C07C 303/30; C07C 19/08; C07C 43/126

(57) ABSTRACT

An object is to provide a method for producing a fluorine-containing compound by using an easily available compound under relatively mild reaction conditions to produce a fluorine-containing compound, a fluorine-containing compound suitably used in the production method, and a fluorine-containing compound obtained by the production method.

There is provided a method for producing a fluorine-containing compound, the method including: reacting a compound having a partial structure represented by the following formula (a) with a Grignard reagent in the presence of a transition metal compound.

—C(—R^{a})(—R^{b})—CH₂-L Formula (a)
where, in the formula, R^{a} is a fluorine atom or a fluoroalkyl group, R^{b} is a hydrogen atom or a fluoroalkyl group, and L is a sulfonate group.

### INCORPORATION BY REFERENCE

This application is based upon and claims the benefit of priority from Japanese Patent Application 2021-034906 filed on Mar. 5, 2021, and PCT application No. PCT/JP2022/008822 filed on Mar. 2, 2022, the disclosure of which is incorporated herein in its entirety by reference.

### BACKGROUND

The present invention relates to a method for producing a fluorine-containing compound and a fluorine-containing compound.

Fluorine compounds are used in various fields such as agricultural chemicals, pharmaceuticals, and functional materials, and it is required to synthesize various structures by a simpler method.

Various studies have been made on a method for synthesizing a compound having a structure in which an alkyl group is bonded to a fluoroalkyl group.

For example, Japanese Unexamined Patent Application Publication No. 2018-43940 discloses a method for producing a fluorine-containing compound by adding a perfluoroalkyl bromide to an olefin compound by a radical reaction.

Examples of International Patent Publication No. WO 2018/228975 disclose a method of reacting a Grignard reagent with R^{f}—CF₂CH₂CH₂—I (R^{f} is a perfluoroalkyl group) which is an electrophile.

In addition, Teruo Umemoto, “Electrophilic Perfluoroalkylating Agents”, Chem. Rev. 1996, 96, 1757-1777 discloses a compound represented by the following formula as an electrophilic perfluoroalkylating agent.

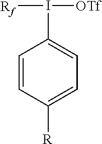

where R_{f} is n-CₘF₂ₘ₊₁, Tf is SO₂CF₃, and R is H or F.

### SUMMARY

The technique of Japanese Unexamined Patent Application Publication No. 2018-43940 is not suitable for synthesis of a compound having a carbon-carbon double bond because an olefin is reacted, and the type of electrophile is limited. In addition, since the product can further undergo a radical reaction to be telomerized, various types of by-products are generated.

The electrophile of International Patent Publication No. WO 2018/228975 was not easily available.

In addition, the electrophilic perfluoroalkylating agent of Teruo Umemoto, “Electrophilic Perfluoroalkylating Agents”, Chem. Rev. 1996, 96, 1757-1777 as an electrophile requires a multi-step process for synthesis, has a low yield, and is expensive.

An object of the present invention is to provide a method for producing a fluorine-containing compound by using an easily available compound under relatively mild reaction conditions to produce a fluorine-containing compound, a fluorine-containing compound suitably used in the production method, and a fluorine-containing compound obtained by the production method.

As a configuration for achieving the above object, the present invention relates to the following [1] to [15].
[1] A method for producing a fluorine-containing compound, the method including:
reacting a compound having a partial structure represented by the following formula (a) with a Grignard reagent in the presence of a transition metal compound.

—C(—R^{a})(—R^{b})—CH₂-L Formula (a)
where, in the formula,
R^{a} is a fluorine atom or a fluoroalkyl group,
R^{b} is a hydrogen atom or a fluoroalkyl group, and
L is a sulfonate group.
[2] The method for producing a fluorine-containing compound according to [1], in which the compound having a partial structure represented by the formula (a) is a compound represented by the following formula (A1) or (A2).

G¹-C(—R^{a})(—R^{b})—CH₂-L Formula (A1)

L-CH₂—{C(—R^{a})(—R^{b})-G²-}ₙC(—R^{a})(—R^{b})—CH₂-L Formula (A2)
where, in the formula,
R^{a} is a fluorine atom or a fluoroalkyl group, and when there are a plurality of R^{a}'s, the R^{a}'s may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
L is a sulfonate group, and a plurality of L's may be the same as or different from each other in the formula (A2), and
n is 0 or 1.
[3] The method for producing a fluorine-containing compound according to [2], in which G¹ is a monovalent group having a (poly)oxyfluoroalkylene chain or a perfluoroalkyl group in the formula (A1).
[4] The method for producing a fluorine-containing compound according to [2], in which n is 0, or n is 1 and G² is a divalent group having a (poly)oxyfluoroalkylene chain, a single bond, or a perfluoroalkylene group in the formula (A2).
[5] The method for producing a fluorine-containing compound according to any one of [1] to [4], in which the Grignard reagent is represented by the following formula (B).

R—MgX Formula (B)
where, in the formula,
R is a hydrocarbon group which may have a substituent or a heteroatom in the carbon chain, and
X is a halogen atom.
[6] The method for producing a fluorine-containing compound according to [5], in which the Grignard reagent is represented by the following formula (B1).

R¹—CH₂—MgX Formula (B1)
where, in the formula,
R¹ is a hydrogen atom or a hydrocarbon group which may have a substituent or a heteroatom in the carbon chain, and
X is a halogen atom.
[7] The method for producing a fluorine-containing compound according to any one of [1] to [6], in which L is a triflate group.
[8] The method for producing a fluorine-containing compound according to any one of [1] to [7], in which the transition metal compound contains copper.
[9] A fluorine-containing compound represented by the following formula (A1) or (A2).

G¹-C(—R^{a})(—R^{b})—CH₂-L Formula (A1)

L-CH₂—{C(—R^{a})(—R^{b})-G²-}ₙC(—R^{a})(—R^{b})—CH₂-L Formula (A2)
where, in the formula,
R^{a} is a fluorine atom or a fluoroalkyl group, and when there are a plurality of R^{a}'s, the R^{a}'s may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
L is a sulfonate group, and a plurality of L's may be the same as or different from each other in the formula (A2), and
n is 0 or 1.
[10] The fluorine-containing compound according to [9], in which G¹ is a monovalent group having a (poly)oxyfluoroalkylene chain or a perfluoroalkyl group in the formula (A1).
[11] The fluorine-containing compound according to [9], in which n is 0, or n is 1 and G² is a divalent group having a (poly)oxyfluoroalkylene chain, a single bond, or a perfluoroalkylene group in the formula (A2).
[12] The fluorine-containing compound according to any one of [9] to [11], in which L is a triflate group.
[13] A fluorine-containing compound represented by the following formula (C1) or (C2).

G¹-C(—R^{a})(—R^{b})—CH₂—R Formula (C1)

R—CH₂—{C(—R^{a})(—R^{b})-G²-}ₙC(—R^{a})(—R^{b})—CH₂—R Formula(C2)
where, in the formula,
R^{a} is a fluorine atom or a fluoroalkyl group, and when there are a plurality of R^{a}'s, the R^{a}'s may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
R is a hydrocarbon group which may have a substituent or a heteroatom in the carbon chain, and
n is 0 or 1.
[14] The fluorine-containing compound according to [13], in which G¹ is a monovalent group having a (poly)oxyfluoroalkylene chain or a perfluoroalkyl group in the formula (C1).
[15] The fluorine-containing compound according to [13], in which n is 0, or n is 1 and G² is a divalent group having a (poly)oxyfluoroalkylene chain, a single bond, or a perfluoroalkylene group in the formula (C2).

The above and other objects, features and advantages of the present disclosure will become more fully understood from the detailed description given hereinbelow which are given by way of illustration only, and thus are not to be considered as limiting the present disclosure.

According to the present invention, there are provided a method for producing a fluorine-containing compound by using an easily available compound under relatively mild reaction conditions to produce a fluorine-containing compound, a fluorine-containing compound suitably used in the production method, and a fluorine-containing compound obtained by the production method.

### DESCRIPTION OF EMBODIMENTS

In the present specification, the partial structure represented by the formula (a) is referred to as a partial structure (a). In addition, the compound represented by the formula (A1) is referred to as a compound (A1). The same applies to compounds represented by other formulae and the like.

The “(poly)oxyfluoroalkylene” is a generic term for oxyfluoroalkylene and polyoxyfluoroalkylene.

The perfluoroalkyl group means a group in which all hydrogen atoms of the alkyl group are substituted with fluorine atoms. In addition, the fluoroalkyl group is a generic term for a combination of a partial fluoroalkyl group and a perfluoroalkyl group. The partial fluoroalkyl group is an alkyl group in which one or more hydrogen atoms are substituted with a fluorine atom and which has one or more hydrogen atoms.

That is, the fluoroalkyl group is an alkyl group having one or more fluorine atoms.
“to” indicating a numerical range means that the numerical values stated before and after “to” are included as a lower limit value and an upper limit value.

### [Method for Producing Fluorine-Containing Compound]

A method for producing a fluorine-containing compound according to the present invention (hereinafter referred to as “present production method”) includes reacting a compound having a partial structure represented by the following formula (a) (hereinafter referred to as a compound (A)) with a Grignard reagent in the presence of a transition metal compound.

—C(—R^{a})(—R^{b})—CH₂-L Formula (a)
where, in the formula,
R^{a} is a fluorine atom or a fluoroalkyl group,
R^{b} is a hydrogen atom or a fluoroalkyl group, and
L is a sulfonate group.

When the Grignard reagent is represented by the following formula (B), the reaction is represented by the following scheme (1).

R—MgX Formula (B)
where, in the formula,
R is a hydrocarbon group which may have a substituent or a heteroatom in the carbon chain, and
X is a halogen atom.

—C(—R^{a})(—R^{b})—CH₂-L+R—MgX→—C(—R^{a})(—R^{b})—CH₂—R Scheme (1)
where, each reference sign in Scheme (1) is as described above.

In the present production method, the reaction of the scheme (1) can be performed under relatively mild reaction conditions by using a sulfonate group as a leaving group L of the partial structure (a) that reacts with a Grignard reagent. Hereinafter, each configuration of the present production method will be described in detail.

L of the partial structure (a) is a sulfonate group (—O—SO₂—R²), and is eliminated by reaction with a Grignard reagent. R² is an organic group. Specific examples of the sulfonate group include a tosylate group (OTs), a mesylate group (OMs), a triflate group (OTf), and a nonaflate group (ONf). Among them, a triflate group is preferable from the viewpoint of the reaction yield of the scheme (1).

Examples of the fluoroalkyl group in R^{a} and R^{b} include a linear or branched alkyl group. The number of carbon atoms in the fluoroalkyl group is preferably 1 to 18, and from the viewpoint of ease of synthesis of the compound (A) and the like, the number of carbon atoms is more preferably 1 to 12, and still more preferably 1 to 6. Specific examples of the fluoroalkyl group include CF₃—, CHF₂—, CH₂F—, CF₃CF₂—, CF₃CF₂CF₂—, CF₃CF(CF₃)—, and CF₃CF₂CF(—CF₂CF₃)—. In addition, the fluoroalkyl groups of R^{a} and R^{b} may be the same as or different from each other.

R^{a} is preferably a fluorine atom or a fluoroalkyl group having 1 to 6 carbon atoms, more preferably a fluorine atom or a perfluoroalkyl group having 1 to 6 carbon atoms, and still more preferably a fluorine atom from the viewpoint of ease of synthesis of the compound (A) and the like.

In addition, R^{b} is preferably a hydrogen atom or a fluoroalkyl group having 1 to 6 carbon atoms, more preferably a fluoroalkyl group having 1 to 6 carbon atoms, and still more preferably a perfluoroalkyl group having 1 to 6 carbon atoms, from the viewpoint of ease of synthesis of the compound (A) and the like.

The compound having the partial structure (a) is a compound having one or more partial structures (a). The number of the partial structures (a) in the compound (A) is preferably 1 to 6, more preferably 1 to 4, and still more preferably 1 and 2 from the viewpoint of reaction yield.

The structure of the compound (A) may be appropriately selected according to the application and the like of the fluorine-containing compound obtained by the present production method.

Examples of the compound (A) having n5 partial structures (a) include a compound represented by the following formula (An5).

G(-C(—R^{a})(—R^{b})—CH₂-L)ₙ₅ Formula (An5)
where, in the formula,
G is a hydrogen atom (where n5=1) or an n5-valent organic group,
n5 is an integer of 1 or more,
R^{a}, R^{b}, and L are as described above, and when there are a plurality of R^{a}'s, R^{b}'s, or L's, the R^{a}'s, R^{b}'s, or L's may be the same as or different from each other.

The organic group in G is a group containing one or more carbon atoms. Examples of the organic group include a hydrocarbon group which may have a substituent or a heteroatom or a bond other than the hydrocarbon group in the carbon chain or at the terminal bonded to the partial structure (a).

Examples of the hydrocarbon group include a linear or branched alkyl group, a cycloalkyl group, an aryl group, and a combination thereof. The hydrocarbon group may have a double bond or a triple bond in the carbon chain. Examples of the combination include a combination in which an alkyl group and an aryl group are bonded directly, via a heteroatom, or via a bond other than a hydrocarbon group.

Examples of the heteroatom include an oxygen atom, a nitrogen atom, a sulfur atom, and a silicon atom.

The heteroatom may constitute a part of the ring structure. In addition, among the heteroatoms, a nitrogen atom, a sulfur atom, and a silicon atom may constitute a branch point bonded to three or more carbon atoms.

Examples of the bond other than the hydrocarbon group include an amide bond, a urea bond, and a urethane bond.

Examples of the substituent that the hydrocarbon group may have include a halogen atom, a hydroxy group, an amino group, a nitro group, and a sulfo group, and from the viewpoint of the stability of the compound in the present production method, a halogen atom is preferable, and among them, a fluorine atom is more preferable.

When the organic group has a ring structure such as a cycloalkyl group or an aryl group, examples of the ring structure include a 3- to 8-membered aliphatic ring, a 6- to 8-membered aromatic ring, a 3- to 8-membered heterocyclic ring, and a condensed ring consisting of two or more of these rings, and a ring structure represented by the following formula is preferable.

The ring structure may have, as a substituent, a halogen atom, an alkyl group which may have an ether bond, a cycloalkyl group, an alkenyl group, an allyl group, an alkoxy group, an oxo group, or the like.

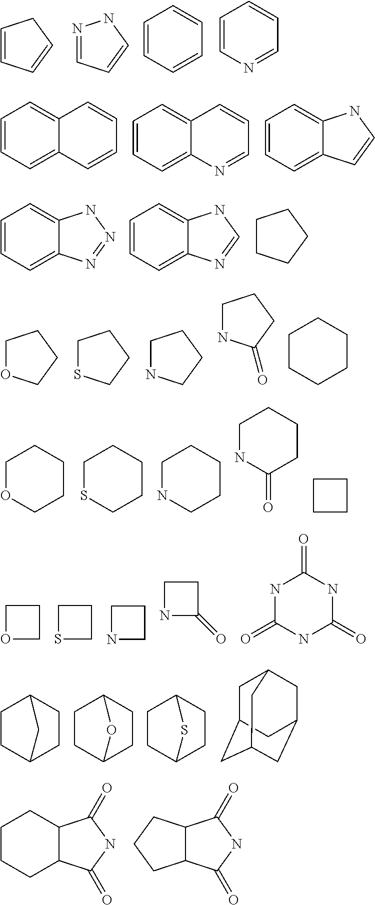

Preferred specific examples of the compound containing a ring structure among the compounds (A) include the following compounds.

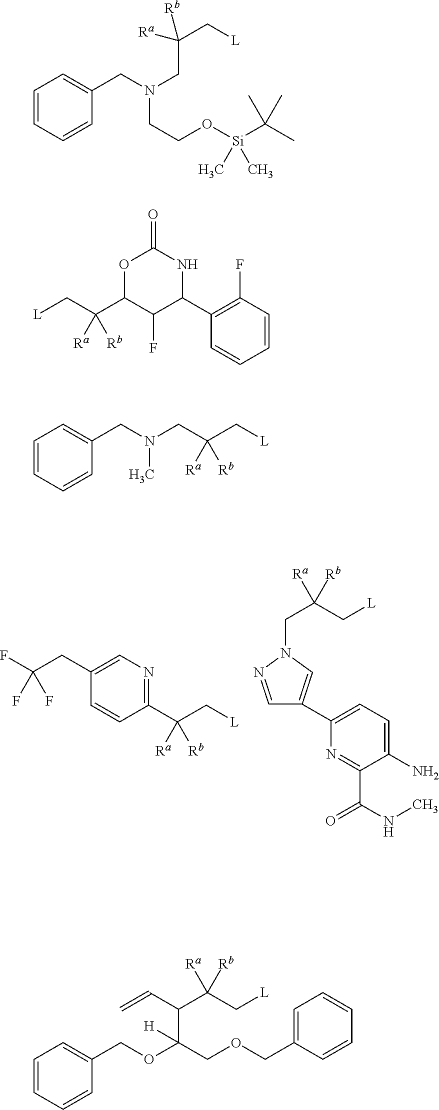

where, R^{a}, R^{b}, and L are as described above.

From the viewpoint of increasing the yield of the present production method, the compound (A) is preferably a compound represented by the following formula (A1) or (A2).

G¹-C(—R^{a})(—R^{b})—CH₂-L Formula (A1)

L-CH₂—{C(—R^{a})(—R^{b})-G²-}ₙC(—R^{a})(—R^{b})—CH₂-L Formula (A2)
where, in the formula,
R^{a}, R^{b}, and L are as described above, and when there are a plurality of R^{a}'s, R^{b}'s, or L's, the R^{a}'s, R^{b}'s, or L's may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group, and
n is 0 or 1.

The number of carbon atoms in the alkyl group or fluoroalkyl group of G¹ is preferably 1 to 30, more preferably 1 to 20, still more preferably 1 to 10, and particularly preferably 1 to 6 from the viewpoint of increasing the yield of the present production method.

The monovalent group having a (poly)oxyfluoroalkylene chain in G¹ is a fluoroalkyl group having —O— at a terminal bonded to C(—R^{a})(—R^{b}), —O— between carbon-carbon atoms of a carbon chain having 2 or more carbon atoms, or both of them in the formula (A1). From the viewpoint of ease of production and the like, G¹ preferably has a structure represented by the following formula (G1-1).

R^{f0}O—[(R^{f1}O)ₘ₁(R^{f2}O)ₘ₂(R^{f3}O)ₘ₃(R^{f4}O)ₘ₄(R^{f5}O)ₘ₅(R^{f6}O)ₘ₆]—(R^{f7})ₘ₇— Formula (G1-1)
where
R^{f0} is a fluoroalkyl group having 1 to 20 carbon atoms,
R^{f1} is a fluoroalkylene group having 1 carbon atom,
R^{f2} is a fluoroalkylene group having 2 carbon atoms,
R^{f3} is a fluoroalkylene group having 3 carbon atoms,
R^{f4} is a fluoroalkylene group having 4 carbon atoms,
R^{f5} is a fluoroalkylene group having 5 carbon atoms,
R^{f6} is a fluoroalkylene group having 6 carbon atoms,
R^{f7} is a fluoroalkylene group having 1 to 6 carbon atoms,
m1, m2, m3, m4, m5, and m6 each independently represent an integer of 0 or 1 or more, m7 is an integer of 0 or 1, and m1+m2+m3+m4+m5+m6+m7 is an integer of 0 to 200.

Note that the bonding order of (R^{f1}O) to (R^{f6}O) in the formula (G1-1) is random.

m1 to m6 in the formula (G1-1) represent the number of (R^{f1}O) to (R^{f6}O), respectively, and do not represent arrangement. For example, (R^{f5}O)ₘ₅ represents that the number of (R^{f5}O) is m5, and does not represent a block arrangement structure of (R^{f5}O)ₘ₅. Similarly, the order of description of (R^{f1}O) to (R^{f6}O) does not represent the bonding order of the respective units.

When m7 is 0, the terminal of G¹ bonded to C(—R^{a})(—R^{b}) is —O—. When m7 is 1, the terminal of G¹ bonded to C(—R^{a})(—R^{b}) is a carbon atom (carbon atom at the terminal of R^{f7}).

Specific examples of G¹ include CH₃—, CH₃CH₂—, CH₃CH₂CH₂—, CH₃CH₂CH₂CH₂—, CH₃CH₂CH₂CH₂CH₂—, CH₃CH₂CH₂CH₂CH₂CH₂—, CF₃—, CF₃CF₂—, CF₃CF₂CF₂—, CF₃CF₂CF₂CF₂—, CF₃CF₂CF₂CF₂CF₂—, CF₃CF₂CF₂CF₂CF₂CF₂—, CF₃CF₂CF₂—O—[(CF₂—O)ₘ₁(CF₂CF₂—O)ₘ₂]—, CF₃CF₂CF₂—O—CF₂CF₂—O—[(CF₂—O)ₘ₁(CF₂CF₂—O)ₘ₂]—, CF₃—O(—CF₂CF₂—O—CF₂CF₂CF₂CF₂—O)ₘ₈—CF₂CF₂—O—CF₂CF₂—, F(—CF₂CF₂CF₂—O)ₘ₃—CF₂—, CF₃—CF(—CF₃)—CF₂—O—, CF₃—CF(—CF₂CF₃)—CF₂—O—, and CF₃CF₂CF₂—O—(CF(—CF₃)—CF₂—O—)ₘ₉- (where m8 and m9 are integers of 1 to 100).

In the present production method, from the viewpoint of yield or the like, G¹ is a monovalent group having a (poly)oxyfluoroalkylene chain or a perfluoroalkyl group in the formula (A1).

The number of carbon atoms in the alkylene group or fluoroalkylene group of G² is preferably 1 to 30, more preferably 1 to 20, still more preferably 1 to 10, and particularly preferably 1 to 6 from the viewpoint of increasing the yield of the present production method.

The divalent group having a (poly)oxyfluoroalkylene chain in G² is a fluoroalkylene group having —O— at two terminals bonded to C(—R^{a})(—R^{b}) each independently, or —O— between carbon-carbon atoms of a carbon chain having 2 or more carbon atoms, or is a combination thereof in the formula (A2). From the viewpoint of ease of production and the like, G² preferably has a structure represented by the following formula (G2-1).

—(O)ₘ₀—[(R^{f1}O)ₘ₁(R^{f2})ₘ₂(R^{f3}O)ₘ₃(R^{f4}O)ₘ₄(R^{f5}O)ₘ₅(R^{f6}O)ₘ₆]—(R^{f7})ₘ₇— Formula (G2-1)

Here, m0 is an integer of 0 or 1, and R^{f1}, R^{f2}, R^{f3}, R^{f4}, R^{f5}, R^{f6}, R^{f7}, m1, m2, m3, m4, m5, m6, and m7 are the same as those in the G¹. Note that the bonding order of (R^{f1}O) to (R^{f6}O) in the formula (G2-1) is random and is as described in the above formula (G1-1).

When m7 is 0, the terminal of G² bonded to C(—R^{a})(—R^{b}) is —O—. When m7 is 1, one terminal of G² bonded to C(—R^{a})(—R^{b}) is a carbon atom (carbon atom at the terminal of R^{f7}). In addition, when m0 is 1, one terminal of G² bonded to C(—R^{a})(—R^{b}) is —O—. When m0 is 0, one terminal of G² bonded to C(—R^{a})(—R^{b}) is a carbon atom (carbon atom at any terminal of R^{f1} to R^{f7}). Note that m0 and m7 are each independently 0 or 1.

Specific examples of G² include —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CF₂—, —CF₂CF₂—, —CF₂CF₂CF₂—, —CF₂CF₂CF₂CF₂—, —CF₂CF₂CF₂CF₂CF₂—, —CF₂CF₂CF₂CF₂CF₂CF₂—, —O—[(CF₂—O)ₘ₁(CF₂CF₂—O)ₘ₂]—, —CF(—CF₃)—CF₂—O—, —CF(—CF₂CF₃)—CF₂—O—, and —O—CF(—CF₂CF₃)—CF₂—O—CF₂—.

In addition, in the formula (A2), when n is 0, the compound (A) is L-CH₂—C(—R^{a})(—R^{b})—CH₂-L. Further, in the formula (A2), when n is 1 and G² is a single bond, the compound (A) is L-CH₂—C(—R^{a})(—R^{b})—C(—R^{a})(—R^{b})—CH₂-L.

In the present production method, from the viewpoint of yield or the like, in which n is 0, or n is 1 and G² is a divalent group having a (poly)oxyfluoroalkylene chain, a single bond, or a perfluoroalkylene group in the formula (A2).

Preferred specific examples of the compound (A) include the following compounds.

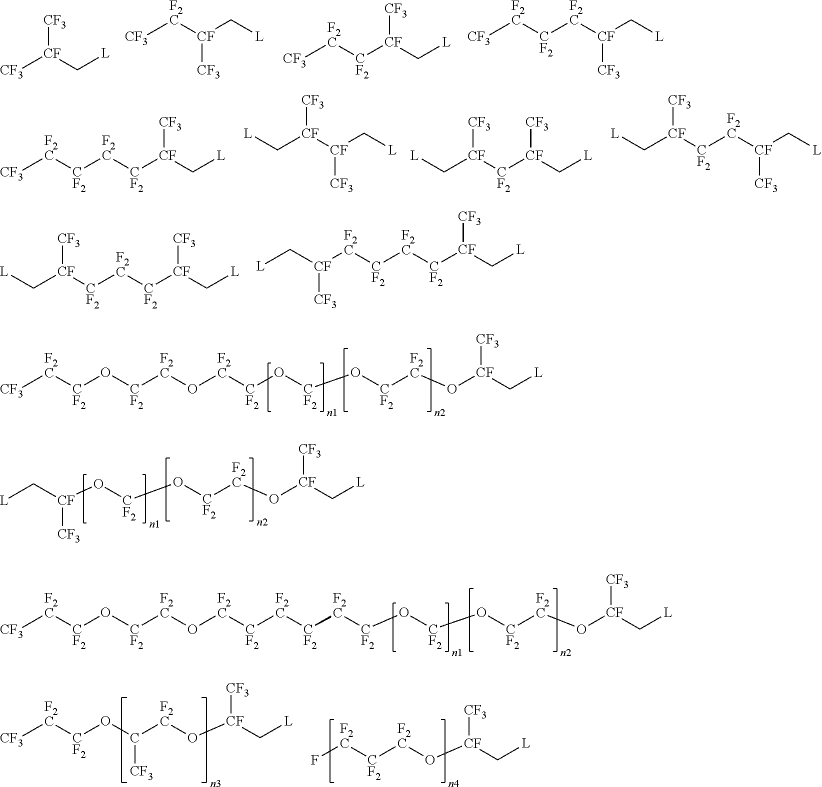

Here, n1, n2, n3, and n4 are integers of 1 to 100.

The compound (A) can be produced, for example, by a method in which a compound represented by the following formula (A1-2) or (A2-2) is reacted with trifluoromethanesulfonic anhydride, tosyl chloride, mesyl chloride, or the like in the presence of an organic amine compound such as triethylamine or pyridine to sulfonate the compound.

G¹-C(—R^{a})(—R^{b})—CH₂—OH Formula (A1-2)

HO—CH₂—{C(—R^{a})(—R^{b})-G²-}ₙC(—R^{a})(—R^{b})—CH₂—OH Formula (A2-2)

However, R^{a}, R^{b}, G¹, G², and n in the formula are as described above.

The Grignard reagent may be any reagent that can react with the partial structure (a). In the present production method, the Grignard reagent is preferably a compound represented by the following formula (B) from the viewpoint of suppressing side reactions and the like.

R—MgX Formula (B)
where, in the formula,
R is a hydrocarbon group which may have a substituent or a heteroatom in the carbon chain, and
X is a halogen atom.

R can be appropriately selected and used from those having a desired structure to be introduced into the compound (A).

The hydrocarbon group in R may have a heteroatom, a substituent, or a double bond or a triple bond with a group including a linear alkyl group, a branched alkyl group, a cycloalkyl group, an aryl group, and a combination thereof as a basic skeleton.

Examples of the heteroatom include a nitrogen atom (N), an oxygen atom (O), a sulfur atom (S), and a silicon atom (Si), and N, O, or S is preferable from the viewpoint of stability of the compound. The substituent is preferably a fluorine atom. From the viewpoint of improving the yield in the present production method, the number of carbon atoms of R is preferably 1 to 30, more preferably 1 to 20, and still more preferably 1 to 15.

From the viewpoint of reactivity, the halogen atom in X is preferably a chlorine atom, a bromine atom, or an iodine atom, and more preferably a chlorine atom or a bromine atom.

Examples of such a Grignard reagent include a primary alkyl Grignard reagent in which a carbon atom bonded to magnesium is a primary carbon atom, such as methylmagnesium chloride, ethylmagnesium chloride, or allylmagnesium chloride; secondary alkyl Grignard reagents such as isopropyl magnesium chloride; tertiary alkyl Grignard reagents such as tert-butyl magnesium chloride; and aryl Grignard reagents such as phenylmagnesium chloride, and vinylmagnesium chloride.

In the present production method, the Grignard reagent is preferably a Grignard reagent represented by the following formula (B1) from the viewpoint of obtaining a target product with high yield.

R¹—CH₂—MgX Formula (B1)
where, in the formula,
R¹ is a hydrogen atom or a hydrocarbon group which may have a substituent or a heteroatom in the carbon chain, and
X is a halogen atom. R¹ is preferably a residue obtained by removing —CH₂ from R.

Since the carbon atom bonded to magnesium is a primary carbon atom, the present production method can be performed under relatively mild reaction conditions.

Preferred specific examples of the formula (B1) include the following.

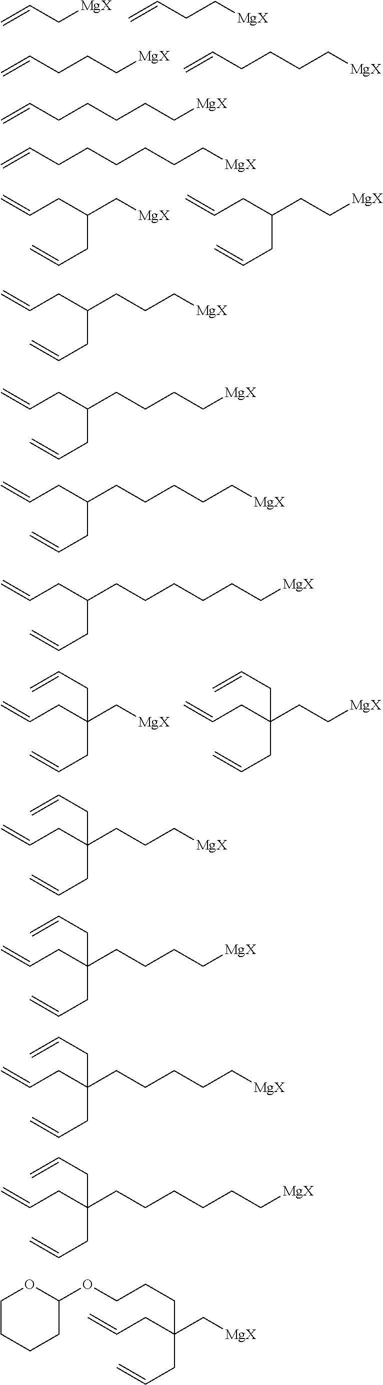

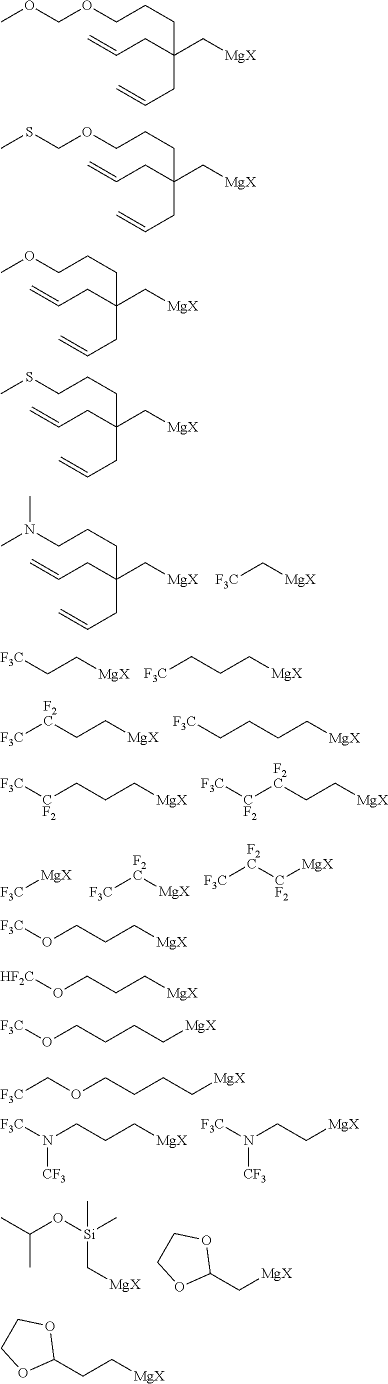

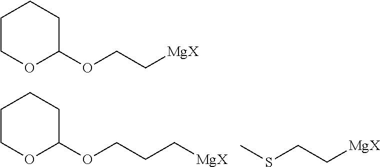

The Grignard reagent can be produced, for example, by reacting the following formula (B2) with metallic magnesium. In addition, a commercially available product having a desired structure may be used.

R—X Formula (B2)
where, R and X are as described above.

In the reaction of Scheme (1), from the viewpoint of improving the yield of the target product, the amount of the Grignard reagent used is preferably 1 equivalent to 30 equivalents, more preferably 3 equivalents to 20 equivalents, and still more preferably 5 equivalents to 15 equivalents, with respect to the total number of leaving groups L of the compound (A).

The transition metal compound can be appropriately selected and used from known catalysts used in the Grignard reaction. As the transition metal compound, a compound containing elements from groups 3 to 12 of the periodic table as transition metals is preferable, and among them, a compound containing elements from groups 8 to 11 is preferable. Among them, the elements from groups 8 to 11 preferably include one or more elements selected from copper, nickel, palladium, cobalt, and iron, and more preferably include copper.

When the transition metal compound contains copper, the copper may be any of zerovalent, monovalent, divalent, and trivalent compounds, and among them, monovalent or divalent copper salts or complex salts are preferable from the viewpoint of catalytic ability. Further, copper chloride is more preferable from the viewpoint of easy availability and the like. As copper chloride, either CuCl or CuCl₂ can be suitably used. In addition, copper chloride may be an anhydride or a hydrate, but copper chloride anhydride is more preferable from the viewpoint of catalytic ability. The amount of the transition metal compound used is, for example, 0.1 to 50% by mol, preferably 1 to 30% by mol, and more preferably 2 to 20% by mol with respect to the total number of leaving groups L of the compound (A).

In the reaction of the present production method, a ligand may be used in combination with a transition metal compound as a catalyst as necessary. By using the ligand, the yield of the target product is improved. On the other hand, in the present production method, since a sufficient yield can be obtained without using a ligand, the ligand may not be used.

Examples of the ligand include 1,3-butadiene, phenylpropyne, and tetramethylethylenediamine (TMEDA). When a ligand is used, the amount used is preferably 0.01 and 2.0 equivalents, more preferably 0.1 to 1.2 equivalents, relative to the total number of leaving groups L of the compound (A) from the viewpoint of improving the yield of the target product.

In addition, the reaction of the present production method is usually performed in a solvent. The solvent can be appropriately selected and used from among solvents capable of dissolving the compound (A) and the Grignard reagent. The solvent may be a single solvent or a mixed solvent of two or more solvents.

For example, when the compound (A) is a compound having a relatively low fluorine atom content (the ratio of fluorine atoms to the molecular weight of the compound molecule), the solvent is not particularly limited as long as the solvent is a solvent inert to the reaction. Among them, the solvent inert to the reaction is preferably an ether-based solvent such as diethyl ether, tetrahydrofuran, or dioxane, and more preferably tetrahydrofuran.

In addition, when the compound (A) is a compound having a relatively high fluorine atom content, a mixed solvent obtained by combining the ether-based solvent and the fluorine-based solvent is preferable.

Examples of the fluorine-based solvent include hydrofluorocarbons (1H,4H-perfluorobutane, 1H-perfluorohexane, 1,1,1,3,3-pentafluorobutane, 1,1,2,2,3,3,4-heptafluorocyclopentane, 2H,3H-perfluoropentane, and the like), hydrochlorofluorocarbons (3,3-dichloro-1,1,1,2,2-pentafluoropropane, 1,3-dichloro-1,1,2,2,3-pentafluoropropane (HCFC-225cb), and the like), hydrofluoroethers (CF₃CH₂OCF₂CF₂H (AE-3000), (perfluorobutoxy)methane, (perfluorobutoxy)ethane, and the like), hydrochlorofluoroolefins ((Z)-1-chloro-2,3,3,4,4,5,5-heptafluoro-1-pentene (HCFO-1437 dycc (Z) form), (E)-1-chloro-2,3,3,4,4,5,5-heptafluoro-1-pentene (HCFO-1437 dycc (E) form), (Z)-1-chloro-2,3,3-trifluoro-1-propene (HCFO-1233 yd (Z) form), (E)-1-chloro-2,3,3-trifluoro-1-propene (HCFO-1233 yd (E) form), and the like), and fluorine-containing aromatic compounds (perfluorobenzene, m-bis(trifluoromethyl)benzene (SR-solvent), p-bis(trifluoromethyl)benzene, and the like).

The present production method can be performed, for example, by preparing a solution containing the compound (A), adding a transition metal compound and a ligand as necessary, and then adding a Grignard reagent solution separately prepared.

The reaction temperature between the compound (A) and the Grignard reagent may be appropriately adjusted according to the combination of the compound (A) and the Grignard reagent. For example, the temperature may be −20° C. to 66° C. (boiling point of tetrahydrofuran), and is preferably −20° C. to 40° C.

According to the present production method, a fluorine-containing compound represented by the following Formula (C1) or (C2) is obtained.

G¹-C(—R^{a})(—R^{b})—CH₂—R Formula (C1)

R—CH₂—{C(—R^{a})(—R^{b})-G²-}ₙC(—R^{a})(—R^{b})—CH₂—R Formula (C2)
where, in the formula, R^{a}, R^{b}, G¹, G², R, and n are as described above, and when there are a plurality of R^{a}'s, R^{b}'s, or Rs, the R^{a}'s, R^{b}'s, or Rs may be the same as or different from each other.

The compound (C1) and the compound (C2) can be used for various applications. In addition, the compounds can be used as intermediates for various compounds. When used as an intermediate, for example, when the compound (C1) or the compound (C2) has a vinyl group, the vinyl group may be hydrosilylated.

The compound (C1) and the compound (C2) may be used as a composition containing other compounds. The other compounds are not particularly limited, and examples thereof include fluorine-containing compounds represented by the following formula (D1) or (D2). When the compound (C1) and the compound (C2) are used as intermediates, the compounds may be used as compositions containing other compounds, or other compounds may be contained in the final product. For example, when the compound (C1) or the compound (C2) has a vinyl group, a composition containing the compound (D1) or the compound (D2) may be further hydrosilylated, or the compound (D1) or the compound (D2) may be contained after the compound (C1) or the compound (C2) is hydrosilylated.

G¹-C(═CF₂)—CH₂—R Formula (D1)

R—CH₂—{C(═CF₂)-G²-}ₙC(═CF₂)—CH₂—R Formula (D2)
where, in the formula, G¹, G², R, and n are as described above, and when there are a plurality of R's, the R's may be the same as or different from each other.

### EXAMPLES

Hereinafter, the present invention will be described in detail using examples, but the present invention is not limited to these examples. In addition, Examples 1, 3 to 10, 12, and 13 are examples, and Examples 2 and 11 are comparative examples.

### Synthesis Example: Synthesis of Compound (A1-1)

2,3,3,4,4,5,5,6,6,7,7,7-Dodecafluoro-2-(trifluoromethyl)-1-heptanol (12 g), dichloromethane (100 mL), and triethylamine (6.0 mL) were added thereto, and the mixture was cooled to 0° C. Trifluoromethanesulfonic anhydride (5.6 mL) was added thereto, and the mixture was stirred at room temperature. After washing the mixture with water, the mixture was dried over sodium sulfate.

After the filtration, the solvent was distilled off, and flash column chromatography was performed using silica gel to obtain 7.3 g of the following compound (A1-1).

The NMR measurement results of Compound (A1-1) are shown below.

¹H-NMR (400 MHz, Chloroform-d) δ 4.41 (d, J=12.3 Hz, 2H).

¹⁹F-NMR (376 MHz, Chloroform-d) δ −70 to −80 (m), −120 to 125 (m), −185 (m).

### Compound (A1-1)

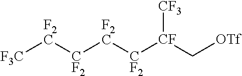

OTf is triflate: —O—S(═O)₂(—CF₃).

### Example 1: Production of Fluorine-Containing Compound (1)

The compound (A1-1) (500 mg), CuCl₂ (21.8 mg) and a 1,3-butadiene THE solution (2.0 M, 0.45 mL) were added, and the mixture was cooled to 10° C. Thereafter, a THF solution of n-butylmagnesium chloride (0.88 M, 9.2 mL) was added dropwise thereto, and the mixture was stirred at room temperature. After cooling the mixture to 0° C., 1 M hydrochloric acid was added, and the mixture was extracted with AE-3000. Sodium sulfate was added, and the mixture was dried, then filtered and concentrated, and flash column chromatography using silica gel was performed to obtain 190.6 mg of the following fluorine-containing compound (1). Note that THE is tetrahydrofuran.

The NMR measurement results of the fluorine-containing compound (1) are shown below.

¹H-NMR (400 MHz, Chloroform-d) δ 2.5 to 1.8 (m, 2H), 1.6 to 1.1 (m, 6H), 1.0 to 0.8 (m, 3H).

¹⁹F-NMR (376 MHz, Chloroform-d) δ −70 to −80 (m), −120 to 125 (m), −165 (m).

### Fluorine-Containing Compound (1)

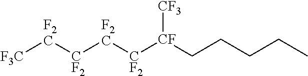

### Examples 2 to 9: Method for Producing Fluorine-Containing Compound (1)

A fluorine-containing compound (1) was produced in the same manner as in Example 1 except that the blending amounts of n-butylmagnesium chloride, 1,3-butadiene, and CuCl₂ were changed as shown in Table 1 below in Example 1.

### Example 10: Method for Producing Fluorine-Containing Compound (1)

A fluorine-containing compound (1) was produced in the same manner as in Example 1 except that CuCl was used instead of CuCl₂ and the blending amount was changed as shown in the following Table 1 in Example 1.

### Example 11: Production of Fluorine-Containing Compound

An attempt was made to produce the fluorine-containing compound (1) using the following compound (X1).

Triphenylphosphine and carbon tetrabromide were added to 2,3,3,4,4,5,5,6,6,7,7,7-dodecafluoro-2-(trifluoromethyl)-1-heptanol, and the mixture was reacted in dichloromethane to synthesize the following compound (X1). However, the compound (X1) was unstable, decomposed during purification, and returned to an alcohol. Therefore, it was found to be unsuitable for synthesis of the fluorine-containing compound (1).

### Compound (X1)

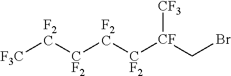

The blending ratio of each component and the yield of the obtained target product in the synthesis of Examples 1 to 10 are shown in Table 1.

Note that e.q. (equivalent) and % by mol in Table 1 are based on the number of triflate groups of the electrophile. A hyphen (-) in the table indicates that nothing is added.

In addition, the yield was determined from the following formula by quantifying the target product by an internal standard method (internal standard: hexafluorobenzene) using ¹⁹F-NMR.

yield=target product/compound (A1-1)×100[%]

TABLE 1

| | | | | Transition metal | | |
|---|---|---|---|---|---|---|
| | | | | compound | | |
| | | n-butyl magnesium | 1,3-butadiene | | Blending | |
| Example | Electrophile | chloride (eq.) | (eq.) | Type | amount (mol %) | Yield (%) |
| 2 | Compound (A1-1) | 1.0 | — | — | — | <1 |
| 3 | Compound (A1-1) | 1.0 | 1.0 | CuCl₂ | 2 | 18 |
| 4 | Compound (A1-1) | 3.0 | 1.0 | CuCl₂ | 6 | 21 |
| 5 | Compound (A1-1) | 5.0 | 1.0 | CuCl₂ | 10 | 36 |
| 6 | Compound (A1-1) | 7.0 | 1.0 | CuCl₂ | 14 | 43 |
| 1 | Compound (A1-1) | 9.0 | 1.0 | CuCl₂ | 18 | 56 |
| 7 | Compound (A1-1) | 9.0 | 1.0 | CuCl₂ | 2 | 52 |
| 8 | Compound (A1-1) | 9.0 | 1.0 | CuCl₂ | 1 | 38 |
| 9 | Compound (A1-1) | 9.0 | — | CuCl₂ | 2 | 48 |
| 10 | Compound (A1-1) | 1.0 | 1.0 | CuCl | 2 | 16 |

As shown in Table 1, according to the production methods of Example 1 and Examples 3 to 10 including reacting the compound (A1-1), which is a compound having a partial structure represented by the formula (a), with a Grignard reagent in the presence of a transition metal compound, it was shown that a target fluorine-containing compound can be synthesized under relatively mild reaction conditions.

Examples 12 and 13 below shows that various compounds can be synthesized by the present production method.

### Example 12: Production of Fluorine-Containing Compound (2)

### Synthesis Example 12-1: Synthesis of Compound (12-1)

2,2′-[(1,1,2,2-Tetrafluoro-1,2-Ethanediyl)bis(oxy)]bis[2,3,3,3-tetrafluoro-1-propanol] (3.85 g), dichloromethane (100 mL), and pyridine (2.2 mL) were added, and the mixture was cooled to 0° C. Trifluoromethanesulfonic anhydride (7.18 g) was added thereto, and the mixture was stirred at room temperature for 3 hours. After washing the mixture with water twice, the mixture was dried over sodium sulfate. After filtration, the solvent was distilled off, and hexane was added. After stirring for 30 minutes, the mixture was filtered and dried under reduced pressure to obtain 2.73 g of the following compound (12-1).

The NMR measurement results of the compound (12-1) are shown below.

¹H-NMR (400 MHz, Chloroform-d) δ 3.86 (m, 4H).

¹⁹F-NMR (376 MHz, Chloroform-d) δ −72 (m), −82 (m), −92 (m), −136 (m).

### Compound (12-1)

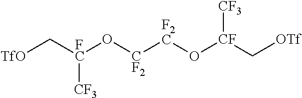

### Synthesis Example 12-2: Synthesis of Fluorine-Containing Compound (2)

Compound (12-1) (0.66 g) and CuCl₂ (2.6 mg) were added, and the mixture was cooled to 10° C., then a THF solution (0.88 M, 10.2 mL) of n-butylmagnesium chloride was added dropwise, and the mixture was stirred at room temperature for 1 hour. After cooling the mixture to 0° C., 1 M hydrochloric acid was added, and the mixture was extracted with AE-3000. Sodium sulfate was added, and the mixture was dried, then filtered and concentrated, and flash column chromatography using silica gel was performed to obtain 0.09 g of the following fluorine-containing compound (2).

The NMR measurement results of the compound (2) are shown below.

¹H-NMR (400 MHz, Chloroform-d) δ 2.5 to 1.8 (m, 4H), 1.6 to 1.1 (m, 12H), 1.0 to 0.8 (m, 6H).

¹⁹F-NMR (376 MHz, Chloroform-d) 6-72 (m), −82 (m), −92 (m), −126 (m).

Compound (2)

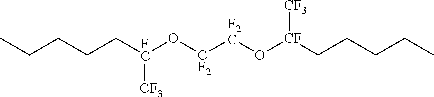

### Example 13: Production of Fluorine-Containing Compound (3)

### Synthesis Example 13-1: Compound (13-1)

As the following compound (13-1), HFPO Alcohol FEOH-2500 manufactured by Sanming Hexafluoro Chemicals was used.

CF₃—CF₂—CF₂—O—(CF(CF₃)—CF₂-0)˜₆—CF(CF₃)—CH₂OH Formula (13-1)

The average value of the number of repeating units n6 is 14.

### Synthesis Example 13-2: Synthesis of Compound (13-2)

4.00 g of the compound (13-1), 2,6-lutidine (0.759 g), and AE-3000 (28.0 g) were added, and the mixture was stirred at 0° C. Trifluoromethanesulfonic anhydride (0.987 g) was added thereto, and then the mixture was stirred at room temperature. After washing the mixture with water, the solvent was distilled off, and flash column chromatography was performed using silica gel to obtain 3.73 g of the following compound (13-2).

CF₃—CF₂—CF₂—O—(CF(CF₃)—CF₂—O)˜₇—CF(CF₃)—CH₂OTf Formula (13-2)

The average value of the number of repeating units n7 is 14, and OTf is triflate: —O—S(═O)₂(—CF₃).

NMR spectrum of the compound (13-2);

¹H-NMR (400 MHz, Chloroform-d) δ 4.95 (m, 2H).

¹⁹F-NMR (376 MHz, Chloroform-d) δ −80 to −85 (m), −131.5 (m), −136 (m).

### Synthesis Example 13-3: Synthesis of Compound (13-3)

Diethyl Diallylmalonate (60.0 g), lithium chloride (23.7 g), water (6.45 g), and dimethyl sulfoxide (263 g) were added, and the mixture was stirred at 160° C. After cooling to room temperature, water was added, and the mixture was extracted with ethyl acetate. Hexane was added to the organic layer, washed with saturated saline, and dried over sodium sulfate. After the filtration, the solvent was distilled off to obtain 39.5 g of the following compound (13-3).

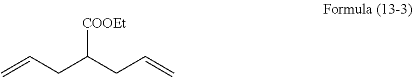

NMR spectrum of the compound (13-3);

¹H-NMR (400 MHz, Chloroform-d) δ (ppm): (ddt, J=17.1, 10.1, 7.0 Hz, 2H), 5.06 to 4.94 (m, 4H), 4.09 (q, J=7.1 Hz, 2H), 2.47 (ddd, J=14.0, 8.0, 6.1 Hz, 1H), 2.33 (dt, J=14.9, 7.5 Hz, 2H), 2.22 (dt, J=14.1, 6.5 Hz, 2H), 1.21 (t, J=7.1 Hz, 3H).

### Synthesis Example 13-4: Synthesis of Compound (13-4)

THF (260 mL) and diisopropylamine (29.8 g) were added, and then the solution was cooled to −78° C. An n-butyl lithium hexane solution (2.76 M, 96.6 mL) was added, and the temperature was raised to 0° C. After stirring, the mixture was cooled to −78° C. to prepare a THF solution of lithium diisopropylamide (LDA). The compound (13-3) (39.5 g) was added to the THF solution, and the mixture was stirred, then allyl bromide (24.1 mL) was added thereto. The temperature was raised to 0° C., 1 M hydrochloric acid (100 mL) was added, and THF was distilled off under reduced pressure. After extraction with dichloromethane, sodium sulfate was added. After the filtration, the solvent was distilled off, and flash column chromatography was performed using silica gel to obtain 45.0 g of the compound (13-4).

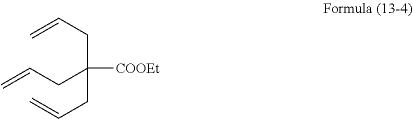

NMR spectrum of the compound (13-4);

¹H-NMR (400 MHz, Chloroform-d) δ (ppm): 5.74 to 5.62 (m, 3H), 5.04 (dd, J=13.6, 1.9 Hz, 6H), 4.10 (q, J=7.1 Hz, 2H), 2.29 (d, J=7.4 Hz, 6H), 1.22 (t, J=7.1 Hz, 3H).

### Synthesis Example 13-5: Synthesis of Compound (13-5)

The compound (13-4) (45.0 g) was dissolved in THF (620 mL), and the solution was cooled to 0° C. A THF solution (104 mL) of lithium aluminum hydride was added thereto, and the mixture was stirred. Water and a 15% sodium hydroxide aqueous solution were added thereto, the mixture was stirred at room temperature, and then diluted with dichloromethane. After the filtration, the solvent was distilled off, and flash column chromatography was performed using silica gel to obtain 31.3 g of the following compound (13-5).

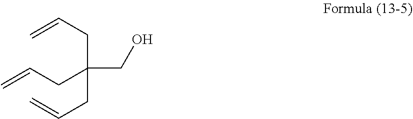

NMR spectrum of the compound (13-5);

¹H-NMR (400 MHz, Chloroform-d) δ (ppm): 5.90 to 5.76 (m, 3H), 5.10 to 5.02 (m, 6H), 3.38 (s, 2H), 2.03 (dt, J=7.5, 1.2 Hz, 6H), 1.45 (s, 1H).

### Synthesis Example 13-6: Synthesis of Compound (13-6)

Acetonitrile (380 mL), the compound (13-5) (31.3 g), triphenylphosphine (64.3 g), and carbon tetrachloride (33.9 g) were added, and the mixture was stirred at 90° C. After concentration, ethyl acetate/hexane was added and stirred. After filtration and concentration, 28.2 g of the following compound (13-6) was obtained by distillation.

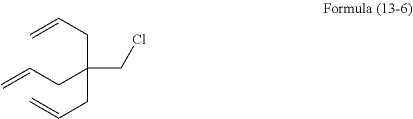

NMR spectrum of the compound (13-6);

¹H-NMR (400 MHz, Chloroform-d) δ (ppm): 5.83 to 5.67 (m, 3H), 5.16 to 5.01 (m, 6H), 3.32 (s, 2H), 2.05 (dt, J=7.5, 1.1 Hz, 6H).

### Synthesis Example 13-7: Synthesis of Compound (13-7)

THF (35 mL) and iodine (0.180 g) were added to magnesium (2.36 g), and the mixture was stirred at room temperature. A THF (35 mL) solution of the compound (13-6) (14.0 g) was added thereto, and the mixture was heated and refluxed for 2 hours to prepare a solution (1.0 M) of the following compound (13-7).

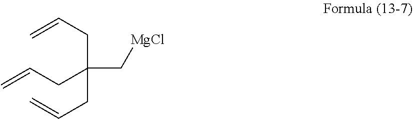

### Synthesis Example 13-8: Synthesis of Fluorine-Containing Compound (3)

CuCl₂ (16.0 mg), 1-phenyl-1-propyne (0.052 g), 1,3-bistrifluoromethylbenzene (24 mL) and the compound (13-1) (2.3 g) were added, and then the compound (13-7) (5.0 mL, 1.0 M) was added. After stirring the mixture at room temperature, the mixture was washed with 1 M hydrochloric acid and dried over sodium sulfate. After filtration, the solvent was distilled off, and AC-6000 was added. After washing the mixture with MeOH, flash column chromatography was performed using silica gel to obtain 0.227 g of the following fluorine-containing compound (3). In addition, AC-6000 is C₆F₁₃C₂H₅.

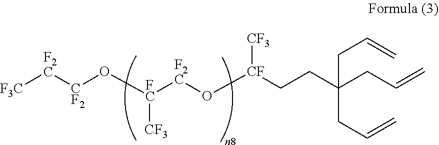

The average value of the number of repeating units n8 is 10.

NMR spectrum of the compound (3);

¹H-NMR (400 MHz, Chloroform-d) δ 1.5 (m), 1.8 to 2.4 (m), 5.0 (m, 6H), 5.8 (m, 3H).

¹⁹F-NMR (376 MHz, Chloroform-d) 6-80 to −85 (m), −94 (m), −105.5 (m), −131.5 (m), −136 (m).

According to the present invention, a fluorine-containing compound used in various fields such as agricultural chemicals, pharmaceuticals, and functional materials can be synthesized under relatively mild reaction conditions using an easily available compound. In addition, for example, by using a Grignard reagent having a carbon-carbon double bond, a double bond can be easily added to the compound (A), and a compound useful as a raw material for synthesizing various compounds can be obtained.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A method for producing a fluorine-containing compound, the method comprising:
reacting a compound having a partial structure represented by the following formula (a) with a Grignard reagent in the presence of a transition metal compound:
—C(—R^{a})(—R^{b})—CH₂-L Formula (a)
where, in the formula,
R^{a} is a fluorine atom or a fluoroalkyl group,
R^{b} is a hydrogen atom or a fluoroalkyl group, and
L is a sulfonate group.

2. The method for producing a fluorine-containing compound according to claim 1, wherein the compound having a partial structure represented by the formula (a) is a compound represented by the following formula (A1) or (A2):
G¹-C(—R^{a})(—R^{b})—CH₂-L Formula (A1)
L-CH₂—{C(—R^{a})(—R^{b})-G²-}ₙC(—R^{a})(—R^{b})—CH₂-L Formula (A2)
where, in the formula,
R^{a} is a fluorine atom or a fluoroalkyl group, and when there are a plurality of R^{a}'s, the R^{a}'s may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
L is a sulfonate group, and a plurality of L's may be the same as or different from each other in the formula (A2), and
n is 0 or 1.

3. The method for producing a fluorine-containing compound according to claim 2, wherein G¹ is a monovalent group having a (poly)oxyfluoroalkylene chain or a perfluoroalkyl group in the formula (A1).

4. The method for producing a fluorine-containing compound according to claim 2, wherein n is 0, or n is 1 and G² is a divalent group having a (poly)oxyfluoroalkylene chain, a single bond, or a perfluoroalkylene group in the formula (A2).

5. The method for producing a fluorine-containing compound according to claim 1, wherein the Grignard reagent is represented by the following formula (B):
R—MgX Formula (B)
where, in the formula,
R is a hydrocarbon group which may have a substituent or a heteroatom in the carbon chain, and
X is a halogen atom.

6. The method for producing a fluorine-containing compound according to claim 2, wherein the Grignard reagent is represented by the following formula (B):
R—MgX Formula (B)
where, in the formula,
R is a hydrocarbon group which may have a substituent or a heteroatom in the carbon chain, and
X is a halogen atom.

7. The method for producing a fluorine-containing compound according to claim 5, wherein the Grignard reagent is represented by the following formula (B1):
R¹—CH₂—MgX Formula (B1)
where, in the formula,
R¹ is a hydrogen atom or a hydrocarbon group which may have a substituent or a heteroatom in the carbon chain, and
X is a halogen atom.

8. The method for producing a fluorine-containing compound according to claim 6, wherein the Grignard reagent is represented by the following formula (B1):
R¹—CH₂—MgX Formula (B1)
where, in the formula,
R¹ is a hydrogen atom or a hydrocarbon group which may have a substituent or a heteroatom in the carbon chain, and
X is a halogen atom.

9. The method for producing a fluorine-containing compound according to claim 1, wherein L is a triflate group.

10. The method for producing a fluorine-containing compound according to claim 2, wherein L is a triflate group.

11. The method for producing a fluorine-containing compound according to claim 1, wherein the transition metal compound contains copper.

12. The method for producing a fluorine-containing compound according to claim 2, wherein the transition metal compound contains copper.

13. A fluorine-containing compound represented by the following formula (A1) or (A2):
G¹-C(—R^{a})(—R^{b})—CH₂-L Formula (A1)
L-CH₂—{C(—R^{a})(—R^{b})-G²-}ₙC(—R^{a})(—R^{b})—CH₂-L Formula (A2)
where, in the formula,
R^{a} is a fluorine atom or a fluoroalkyl group, and when there are a plurality of R^{a}'s, the R^{a}'s may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
L is a sulfonate group, and a plurality of L's may be the same as or different from each other in the formula (A2), and
n is 0 or 1.

14. The fluorine-containing compound according to claim 13, wherein G¹ is a monovalent group having a (poly)oxyfluoroalkylene chain or a perfluoroalkyl group in the formula (A1).

15. The fluorine-containing compound according to claim 13, wherein n is 0, or n is 1 and G² is a divalent group having a (poly)oxyfluoroalkylene chain, a single bond, or a perfluoroalkylene group in the formula (A2).

16. The fluorine-containing compound according to claim 13, wherein L is a triflate group.

17. A fluorine-containing compound represented by the following formula (C1) or (C2):
G¹-C(—R^{a})(—R^{b})—CH₂—R Formula (C1)
R—CH₂—{C(—R^{a})(—R^{b})-G²-}ₙC(—R^{a})(—R^{b})—CH₂—R Formula (C2)
where, in the formula,
R^{a} is a fluorine atom or a fluoroalkyl group, and when there are a plurality of R^{a}'s, the R^{a}'S may be the same as or different from each other,
R^{b} is a hydrogen atom or a fluoroalkyl group, and when there are a plurality of R^{b}'s, the R^{b}'s may be the same as or different from each other,
G¹ is a monovalent group having a (poly)oxyfluoroalkylene chain, a hydrogen atom, an alkyl group, or a fluoroalkyl group,
G² is a divalent group having a (poly)oxyfluoroalkylene chain, a single bond, an alkylene group, or a fluoroalkylene group,
R is a hydrocarbon group which may have a substituent or a heteroatom in the carbon chain, and
n is 0 or 1.

18. The fluorine-containing compound according to claim 17, wherein G¹ is a monovalent group having a (poly)oxyfluoroalkylene chain or a perfluoroalkyl group in the formula (C1).

19. The fluorine-containing compound according to claim 17, wherein n is 0, or n is 1 and G² is a divalent group having a (poly)oxyfluoroalkylene chain, a single bond, or a perfluoroalkylene group in the formula (C2).
